(19) United States
(12) Patent Application Publication
(10) Pub. No.: US 2022/0112445 A1
(43) Pub. Date: Apr. 14, 2022

(54) COMPOSITION, SYSTEM AND METHOD FOR TREATING STAINS AND ODORS

(71) Applicant: Manna Pro Products, LLC, Chesterfield, MO (US)

(72) Inventors: Scott Plasek, Flower Mound, TX (US); Judy Pruitt, Mesquite, TX (US); Christina Bond Burton, Bedford, TX (US); Alisha Farrington, Bedford, TX (US); Robert C. Pearce, III, Arlington, TX (US)

(21) Appl. No.: 17/426,996

(22) PCT Filed: Jan. 28, 2020
(86) PCT No.: PCT/US20/15420

§ 371 Date: Jul. 29, 2021

Related U.S. Application Data
(60) Provisional application No. 62/798,246, filed on Jan. 29, 2019.

(51) Int. Cl. C11D 3/38; C11D 3/00; C11D 3/39; C12N 1/20

(52) U.S. Cl. CPC C11D 3/381; C11D 3/0031; C12R 2001/07; C12N 1/20; C11D 3/3942

(57) ABSTRACT

The present disclosure is directed to a composition and method for treating stains and odors using an oxidizing agent and Bacillus spores. The composition preferably comprises a two part formula, a first part comprising the oxidizing agent and water and optionally an ionic surfactant and fragrance, and a second part comprising a liquid Bacillus spores composition or a dry Bacillus spores powder composition. The preferred Bacillus can include B. licheniformis, B. subtilis, B. pumilus, and B. megaterium spores. The preferred oxidizer is hydrogen peroxide. The parts of the treatment composition are mixed together in-situ at the site of treatment to maintain the viability of the Bacillus spores. The preferred composition and method allow for the combined treatment of stains and odors with an oxidizer and Bacillus spores.

### CROSS-REFERENCE TO RELATED APPLICATIONS

This application claims priority to International Patent Application No. PCT/US2020/015420 filed 28 Jan. 2020, which claims priority to U.S. Provisional Application No. 62/798,246, filed 29 Jan. 2019, the entire contents of which are hereby incorporated herein by reference.

### FIELD OF THE DISCLOSURE

A composition, system and method are disclosed for treating stains and odors using an oxidizer part and Bacillus spores part.

### BACKGROUND OF THE DISCLOSURE

Various treatment products for treating stains and odors are known. These treatment products include oxidizing chemicals that are especially effective on removing stains; however, oxidizing products are also effective on odor reduction. Other treatment products are emulsions containing Bacillus spores that consume and break-down the stain and odor causing agents and are especially effective on reducing odors. Both types of treatment are effective, but have previously only been usable separately because the oxidizing chemicals would destroy the spores in a Bacillus combined treatment product. For example, hydrogen peroxide is a widely used oxidizer and biocide for disinfection. It is a clear, colorless liquid that is miscible with water. Hydrogen peroxide is considered environmentally friendly because it can rapidly degrade into the innocuous products water and oxygen. Hydrogen peroxide demonstrates broad-spectrum efficacy against viruses, bacteria, yeasts, and Bacillus spores. As such, it has been considered in the art to be unsuitable for use in combination with a Bacillus spore treatment or cleaning agent, because the peroxide would kill off the Bacillus before they have a chance to break-down the stain and odor causing agents.

However, it is known in the art that some Bacillus spores are resistant to oxidizing agents. There are several reasons why some Bacillus spores have such resistance and may be better suited for combination with an oxidizer, such as hydrogen peroxide. Over the years, different studies have shown various temperature and length of contact time results for the death or inactivation of Bacillus spores. These results appear to show that other additional factors including pH, catalyst interaction, the species of the Bacillus spores, the method of growth of the spores, temperature of the treatment, and length of contact time are not the same in all situations. Hydrogen peroxide is both bactericidal and sporicidal; however, hydrogen peroxide bactericidal effects and sporicidal effects vary. Higher concentrations of hydrogen peroxide (10 to 30%) and longer contact times are required for sporicidal activity.

Bacillus subtilis strains usually show more resistance to hydrogen peroxide. For example, one study showed no kill of Bacillus subtilis spores at rate of 20 mg/I hydrogen peroxide for 60 minutes. Another study indicated that 10% hydrogen peroxide at room temperature is ineffective against Bacillus subtilis subsp. globigii, and a high concentration (35%) and a high temperature (80° C.) are required for the destruction of these spores. Yet, another study showed that at a concentration of 6% (w/vol), hydrogen peroxide becomes bactericidal, but only slowly sporicidal. However, at 25° C. and levels of between 10 and 20% (w/v), the concentration exponent is about 1.5.

In general, greater activity is seen against vegetative gram-positive bacteria than gram-negative bacteria; however, the presence of catalase or other peroxidases in an organism can increase tolerance in the presence of lower concentrations of hydrogen peroxide. In gram-positive vegetative cells, such as Bacillus species, the peptidoglycan layer of the cell membrane and the associated anionic polymers provide easy access by diffusion through the membrane for the very small hydroxyl radical molecule. Once inside the cell, the hydroxyl radical can damage DNA, nucleic acids, proteins, and lipids. Hydrogen peroxide can directly inactivate enzymes such as gluceraldehyde-3-phosphate, usually by oxidation of thiol groups, attacking exposed sulfhyrdryl groups, attacking exposed double bonds, or polyunsaturated fatty acyl groups in lipids.

The generally proposed mechanism of action of hydrogen peroxide involves its breakdown to yield reactive oxygen species (ROS). ROS can lead to biological damage. Hydrogen peroxide is a source of singlet oxygen, peroxide radical (O₂²), and hydroxyl radical (OH) that are highly reactive and very toxic for bacteria. A radical is an atom or group of atoms with one or more unpaired electrons than can have a positive, negative, or zero charge. Singlet oxygen is highly reactive, and is a more stable radical than the hydroxyl radical. The peroxide ion, or radical, has a single bond between two oxygen atoms resulting in the structural formula of (O—O)⁻² or O₂⁻². The single bond makes peroxide a strong oxidizer. Hydrogen peroxide has the structural formula of H—O—O—H. If the bond between the oxygen atoms, the peroxide bond, is broken; two hydroxyl radicals are formed. Hydroxyl radicals are reactive, cytotoxic and powerful oxidizers which kill vegetative cells. The hydroxyl radical cannot be eliminated by an enzymatic reaction in a cell, and they are the most reactive form of oxygen. Hydroxyl radicals react very quickly. They are short-lived free radicals with a half-life of approximately 10′ sec at 37° C. Hydrogen peroxide can break down in various ways, such as, 2H₂O₂+2H₂O+O₂ and H₂O₂→2H—O.

Detailed studies of spore killing by oxidizing agents have suggested that a major mechanism of spore killing by these chemicals is some type of oxidative damage to the spore's inner membrane. Membrane damage may prevent spores from germinating. Germinating damaged spores may die or lyse. Untreated B. subtilis spores will survive short incubation times at 85° C., and the spores don't lose much DPA. At higher temperatures (>85° C.), spores lose a great deal of DPA, and spores die due to the loss of DPA. Spores killed by hydrogen peroxide contact do not lose large amounts of DPA. Thus, the hydrogen peroxide killing mechanism could be the loss of the ability to maintain the spore core permeability barrier to hydrophilic compounds. Spores treated with a minimally lethal exposure to hydrogen peroxide may suffer some inner membrane damage that only kills the spore if the spore is exposed to another acute stress, such as, high heat.

Still other studies seem to provide evidence that oxidizing agents injure Bacillus spores by damaging the spore's inner membrane. Possible injuries to the spore inner membrane due to hydrogen peroxide exposure include: 1) damage to membrane enzymes that causes problems during spore germination and increases osmotic stress to the germinating spore; 2) damage to membrane proteins, membrane proteins that are required for spore germination: 3) creation of channels or pores in the inner membrane, known as leaky membranes, which releases DPA; and 4) modification of the inner membrane making it more susceptible to heat stress.

Germination Initiated, then Failed. Killing of spores of B. subtilis with hydrogen peroxide caused no release of dipicolinic acid (DPA) and hydrogen peroxide-killed spores were not appreciably sensitized for DPA release upon a subsequent heat treatment. Spores killed by hydrogen peroxide treatment appeared to initiate germination normally, release DPA, and hydrolyze significant amounts of their cortex. However, during germination, spores did not swell, did not accumulate ATP, did not reduce flavin mononucleotide, and the cores of these germinated spores were not accessible to nucleic acid stains. These data indicate that treatment with hydrogen peroxide results in spores in which the core cannot swell properly during spore germination. The results provide further information on the mechanism of killing of spores of Bacillus species by hydrogen peroxide.

Bacillus Sporulation. Endospores produced by members of various Bacillus species are metabolically dormant and extremely resistant to a variety of potential killing agents, including heat, radiation, high pressure, desiccation, enzymes and toxic chemicals such as acids, bases, alkylating agents, aldehydes and oxidizing agents. The Bacillus spore is a highly-evolved structure capable of maintaining the bacterial genome in a protected, viable state for extended periods. Vegetative cells have high cytoplasmic water activity while spores have very low water activity. Spores lose the enzyme activity and macromolecular synthesis present in vegetative cells.

Spores are formed by vegetative cells in response to environmental signals that indicate a limiting factor for vegetative growth, such as exhaustion of an essential nutrient. After a vegetative Bacillus cell undergoes sporulation, the spore becomes well protected against undesirable growth conditions, including elevated temperatures, an absence of substrate, an absence of nutrients, low water activity, or in the case of aerobic Bacillus, too little dissolved oxygen. Bacillus spores germinate and become vegetative cells when the environmental stress is relieved. Hence, endospore-formation is a mechanism of survival rather than a mechanism of reproduction.

Endospores are so named because they are formed intracellularly, although they are eventually released from the mother cell as a free spore. Spores have proven to be the most durable type of cell found in nature, and in their cryptobiotic state of dormancy with no detectable metabolism, they can remain viable for extremely long periods of time. In the dormant spore state, all metabolic procedures stop; preventing reproduction, development, and repair. In a dormant state, a spore retains viability indefinitely.

The variability of cell wall structure that is common in many Gram-positive bacteria does not occur in Bacillus genera. The vegetative cell wall of almost all Bacillus species is made up of a peptidoglycan containing meso-diaminopimelic acid. Bacillus spores show a more complex ultrastructure than is seen in Bacillus vegetative cells. The formation of spores is a complex and highly-regulated form of development in a relatively simple (procaryotic) cell.

Forespore. Bacillus sporulation takes place in a two-compartment sporangium that arises by a process of asymmetric division. The smaller protoplast or core compartment develops into the spore, whereas the larger mother cell nurtures the developing core. Initially, the core (forespore) and mother cell lie side by side; subsequently, the mother cell engulfs the core in a phagocytosis-like process that results in a cell-within-a-cell configuration.

Protoplast. The spore core is surrounded by the core wall, the cortex, and then the spore coat. Between the two membranes, the core (cell) wall, cortex and spore coats are synthesized. The engulfed core is encased in a protective peptidoglycan cortex and protein coat layers. The core wall is composed of the same type of peptidoglycan as the vegetative cell wall. The cortex is composed of a unique peptidoglycan that bears three repeat subunits, always contains DAP, and has very little cross-linking between tetrapeptide chains. The cortex consists largely of peptidoglycan, including a spore-specific muramic lactam.

Spore Coat. The spore coat is a particularly thick protein coat. It is a highly ordered structure consisting of the following three distinct layers: an electron-dense outer coat, a thinner inner coat, and an electron-diffuse undercoat. The outer spore coat represents 30-6) percent of the dry weight of the spore. The spore coat proteins have an unusually high content of cysteine and of hydrophobic amino acids.

Water loss in the spore core during sporulation. As water is removed from the spore, and as it matures, it becomes increasingly heat resistant and more refractile. Refractile refers to the spore appearing white (phase bright) using a phase contrast microscope while vegetative cells or mother cell walls appear a dark blue (phase dark). The mature spore is eventually liberated by lysis of the mother cell. Ultimately the mature spore is freed of the mother cell walls and released into the environment. The spore is now considered a “free spore”. The sporulation process occurs over a 6-7 hour time span.

Dipicolinic Acid production during sporulation. Dipicolinic acid, DPA, is made only during sporulation in the mother cell compartment of the sporulating cell, and the DPA is then taken up into the developing spore. It has been known for many years that one spore specific organic chemical, dipicolinic acid (DPA) or salt calcium dipicolinate (Ca²⁺-DPA) is only found in spores. There is also only one spore-specific enzyme, DPA-synthetase needed for synthesis of DPA. These acids greatly aid in the spores' longevity. Ca²⁺-dipicolinate contributes about 17% to a spore's dry weight.

The major role of the calcium-DPA complex seems to be the reduction of water during the sporulation process. The DPA replacing the water aids in the spore's indestructibility. A key to the incredible longevity of spores is the presence of dipicolinic acid (DPA) and its salt, calcium-dipicolinate, in the living core that contains the spore's DNA, RNA, and protein. The calcium-DPA complex protects a spore against heat stress. Heat can destroy spores by inactivating proteins and DNA, but the process requires a certain amount of water. Since calcium-DPA in the spore limits the amount of water in the spore, the spore is less vulnerable to heat. DPA isolated from spores is nearly always in the Ca²⁺-DPA chelate form. Sometimes the chelate is of another divalent metal, such as, Zn, Mn, Sr, and others; or as a Ca-DPA amino complex. Usual growth media for spore preparation are invariably supplemented with MnCl₂. Bacillus megaterium. B. cereus, and B. subtilis spores containing elevated manganese levels are more resistant to hydrogen peroxide injury. And, in spores of Bacillus subtilis, manganese appears to result in spores with maximal resistance to hydrogen peroxide. Concentration levels of dipicolinic acid and the DNA-protective a/13-type small, acid-soluble spore proteins were the same in spores with either high and low magnesium levels.

Bacillus Spore Resistance. Spores of Bacillus species are extremely resistant to a variety of stress factors including wet or dry heat, UV or g-radiation, or toxic chemicals, including oxidizing agents such as hydrogen peroxide. Bacillus subtilis spores are not as sensitive to hydrogen peroxide as other Bacillus species spores. Spores are highly resistant to many environmental insults because the spore core (cytoplasm) is dehydrated, dormant, and surrounded by multiple protective layers, including a modified layer of peptidoglycan known as the cortex. The cortex functions to maintain dormancy and heat resistance by preventing core rehydration.

DNA protection is a possible factor in the resistance of Bacillus spores to hydrogen peroxide. Spore DNA is highly protected by the spore coat, the low permeability of the spore's inner membrane, the core's low water content, and the saturation of spore DNA by small, acid-soluble proteins (SASP's). The DNA is often not damaged when a spore is killed by oxidizing agents.

Dormant spores of Bacillus can survive for years, possibly because spore DNA is well protected against damage by many different agents. DNA in the core is protected by the saturation of spore DNA with a group of small (low molecular weight), acid-soluble spore proteins (SASP's), which are synthesized in the developing spore, and degraded after completion of spore germination. The structure of both DNA and SASP's alters upon their association, and this causes major changes in the chemical and photochemical reactivity of DNA. SASP's contribute to spore resistance to hydrogen peroxide, but may not be the only factor involved, since the coat and cortex also appear to play roles in spore resistance to hydrogen peroxide. DNA protection is also partly a result of the high level, up to 10% of the dry weight of the spore and approximately 25% of the core dry weight, of Ca²⁺-dipicolinic acid (DPA) which lowers the hydration of the core.

Bacillus spore resistance to hydrogen peroxide differs from strain to strain. Most Bacillus subtilis spores are not as sensitive to hydrogen peroxide. In Bacillus subtilis spores, SASP's comprise up to 20% of the total spore core protein. The multiple a/(3-type) SASP's have been shown to confer resistance to UV radiation, heat, peroxides, and other sporicidal treatments to Bacillus spores.

Other studies indicate that hydrogen peroxide kills spores by damage to proteins, and not damage to DNA. At one time, DNA repair during spore germination was considered a possible key to spore resistance to hydrogen peroxide. However, DNA damage is no longer considered to be lethal to spores, so DNA repair during germination is not required.

Growth temperature is another possible factor in the resistance of Bacillus spores to hydrogen peroxide. Some of the differences in spore resistance may be due to the effects of the sporulation temperature. Spores grown at higher temperatures are almost always more resistant to a number of oxidizing agents. Interestingly, Bacillus spores grown at lower temperatures are invariably more sensitive to oxidizing agents than are spores grown at higher temperatures.

Lower water content is another possible factor in resistance. Spore resistance may be partly due to the low hydration level of the spore core. Ibis low level of water greatly aids in protection against heat stress. Spore heat resistance correlates to increased resistance to oxidizing agents. The low water content of 25% to 50% of the total spore weight protects spore proteins from heat inactivation. Spores that have increased spore core water content have decreased hydrogen peroxide resistance. Some studies show that SASP's may not be as important to spore survival as spore core water content.

Inner membrane content is still another possible factor. The usual targets for peroxide attack on membranes are polyunsaturated fatty acids, but these fatty acids occur at very low levels in spores. It has been noted that spores are somewhat lower in unsaturated fatty acids compared with growing cells. Since spores of strains with very different levels of unsaturated fatty acids in their inner membrane exhibited essentially identical resistance to oxidizing agents, it doesn't appear that oxidation of unsaturated fatty acids by hydrogen peroxide kill and/or damage spores. Perhaps oxidizing agents work by causing oxidative damage to key proteins in the spore's inner membrane. Peroxide treated spores maintain their permeability barrier, thus it is unlikely that significant damage occurs to the spore inner membrane.

Proteins are another possible factor. One alkyl hydroperoxide reductase subunit is present in spores. Results indicate that proteins that play a role in the resistance of growing cells to oxidizing agents play no role in spore resistance. A likely reason for this lack of a protective role for spore enzymes is the inactivity of enzymes within the dormant spore. It is possible that hydrogen peroxide is actively destroyed by an additional catalase that resides in the spore coat itself. For example, SodA gene product protein has been detected in spore coat extracts, and it has been proposed that this enzyme acts in concert with an unidentified catalase to cross-link coat proteins.

Spore coat is also a factor in resistance of Bacillus spores to hydrogen peroxide. A spore coat comprises a major portion of the total Bacillus spore. The spore coat consists largely of protein, with an alkali-soluble fraction made up of acidic polypeptides being found in the inner coat and an alkali-resistant fraction associated with the presence of disulfide-rich bonds being found in the outer coat. The cortex is composed of peptidoglycan. The coat and cortex structures are relevant to the mechanism(s) of resistance presented by Bacillus spores to antiseptics and disinfectants.

Spore coats confer resistance by restricting access of chemicals and enzymes to sensitive targets located further within the spore. The outer spore coat helps protect the dormant spore from enzymes, such as lysozyme. Lysozyme damages 1 cell walls by catalyzing the hydrolysis of 1,4-beta-linkages between N-acetylmuramic acid and N-acetyl-D-glucosamine residues in a peptidoglycan.

The spore coat helps protect spores from mechanical disruption. Resistance to organic solvents and heat seems to be a function of the peptidoglycan cortex which underlies the spore coat. Regardless of the target of hydrogen peroxide in the spore core, it may be that either the spore coat layers serve as a diffusion barrier to hydrogen peroxide, or spore coat proteins act as oxidation targets which decrease the effective hydrogen peroxide concentration before the hydrogen peroxide reaches the target(s) in the spore core. Data indicates that the intact spore coat contributes to the resistance of hydrogen peroxide. Data indicates that the intact spore coat leads to resistance of Bacillus subtilis spores to hydrogen peroxide.

At very high concentrations, hydrogen peroxide can cause a major break up of the spore coat structure and the cortex as well as the core. At lower concentrations, hydrogen peroxide can kill spores as well, just without evident cytological changes.

Spore Germination to a Vegetative Cell. Under appropriate environmental conditions, the spore will germinate into a vegetative cell, and return to its metabolic state of life as it was prior to the cryptobiosis. Germination of a spore is the process by which a dormant spore goes through a number of degradative events in order to become a viable cell. This is a process of interrelated biochemical events that occur in the spore. Germination is induced by nutrients and a variety of non-nutrient agents. Germination inducers are organic compounds that include sugars, dodecylamine, and amino acids. Spores exposed to good growth conditions and specific nutrients will begin the process of hydrolysis of the peptidoglycan cortex by depolymerization of the cortex.

Hydrolysis of the spore's peptidoglycan cortex, mediated by either of two redundant enzymes, occurs. An amino acid can become an activator of certain enzymes which produce germination substances needed to initiate the germination process. For example, L-alanine will induce germination in a Bacillus subtilis spore, L-proline will induce germination in a Bacillus megaterium spore, and inosine will induce germination in a Bacillus cereus spore. After the addition of L-alanine many peptidoglycan structural changes are observed enabling the Bacillus subtilis spore to respond to the germinant. L-alanine is recognized by receptors encoded by homologous tricistronic operons, the gerA, gerB, and gerK in B. subtilis. These operons are encoded by proteins found in the spore's inner membrane. It is the gerA receptor that triggers the spore to germinate with the addition of L-alanine.

The cortex must be removed for the core to grow. The hydrolysis of the cortex, and subsequent core cell wall expansion, results in complete core rehydration, resumption of metabolic activity, macromolecular synthesis, and outgrowth.

Nutrient germinants bind to receptors in the spore's inner membrane. This interaction triggers the release of the high volume of dipicolinic acid and cations from the core of the spore. While the Ca²⁺-DPA pool in the dormant spore is stable over extremely long periods, DPA and its associated cations are released rapidly when spores initiate germination. The germination process triggers many nutrient-receptor interactions, including the release of DPA as well as Ca²⁺. It is the release of DPA which allows the uptake of water into the core of the spore. The DPA and cations are replaced by water. The spore core then swells to a much larger size. Germination is accompanied by a 30% loss in dry weight, partly from loss of the spore coat. The large volumes of SASP's covering the DNA are rapidly degraded during germination.

KatX is the only catalase detectable in the dormant spore. A major function for KatX is to protect germinating spores from hydrogen peroxide damage. It is not surprising that Bacillus spores with a mutant katX gene are hydrogen peroxide sensitive during spore germination. Expression of a katX-lacZ fusion begins at approximately the second hour of sporulation, and >75% of the katX-driven 13-galactosidase is packaged into the mature spore during sporulation. KatA, the major catalase in growing cells of B. subtilis, is not present in spores. Furthermore, the katA gene is not transcribed and producing KatA catalase until at least 20 min after the initiation of spore germination. Thus, KatX appears to be the only catalase that can detoxify hydrogen peroxide early in spore germination. Presumably, after synthesis of KatA, germinating spores will become less hydrogen peroxide sensitive.

Some spores show slower germination times after exposure to hydrogen peroxide while some spores are killed or inactivated by exposure to hydrogen peroxide depending on the treatment conditions. The slow germination of individual hydrogen peroxide treated spores seems to be a result of: 1) 3- to 5-fold longer lag times between germinant addition and initiation of fast release of spores' large DPA deposit; 2) 2- to 10-fold longer times for rapid DPA release, once this process had been initiated; and 3) 3- to 7-fold longer times needed for hydrolysis of the spores' peptidoglycan cortex.

It appears that oxidizing agent treatment may effect spore germination by acting on: A) nutrient germinant receptors in spores' inner membrane; B) components of the DPA release process, possibly SpoVA proteins also in spores' inner membrane, or the cortex-lytic enzyme CwIJ; and C) the cortex-lytic enzyme SIeB, also largely in spores' inner membrane.

Even though it is known that some Bacillus spores show resistance to hydrogen peroxide, the two are not previously known to have been combined into an effective treatment for stains and odors. There is a need for an effective stain and odor treatment product that combines the benefits of an oxidizer with those of a Bacillus spore treatment that can be utilized without destroying the Bacillus spore prior to use, and ultimately, the growing vegetative Bacillus cell during use.

### BRIEF DESCRIPTION OF THE DISCLOSURE

As disclosed herein, a treatment composition is provided for treating stains and odors with a combination of an oxidizing agent and Bacillus A premixed formulated product containing both an oxidizing agent and Bacillus spores is not shelf-life stable since the oxidizing agent adversely affects the Bacillus spores when both are in contact for a prolonged time, i.e. as short a time as a few hours. According to one preferred embodiment, the oxidizing agent and Bacillus spores are stored separately and combined in-situ by the user to provide effective stain and odor treatment on a variety of substrates. The in-situ mixing of the two parts, an oxidizing agent part and a Bacillus spore part, creates an effective treatment for organic stain removal and odor reduction without loss of Bacillus spore count during storage. The resulting application is effective in removing and eliminating the stains and odors via a chemical oxidization reaction and an organic biological bacterial reaction (degradation) process.

According to one preferred embodiment, an oxidizing agent part (also referred to herein as Part One or a First Part) is a a liquid composition comprising an oxidizing agent. Most preferably, the First Part is a water-based emulsion comprising water, hydrogen peroxide, surfactant, and a fragrance.

According to another preferred embodiment, the Bacillus spore part (also referred to herein as Part Two or a Second Part) comprises one or more species of Bacillus in spore form. According to another preferred embodiment, the Bacillus part is a liquid composition comprising one or more species Bacillus spores (also referred to herein as Liquid Part Two or a Liquid Second Part). According to another preferred embodiment, the Bacillus part comprises a dry powder composition comprising one or more species of Bacillus in spore form (also referred to herein as Dry Part Two or a Dry Second Part.

A preferred treatment composition according to the present disclosure comprises a First Part and a Second Part (either a Liquid Second Part or a Dry Second Part) that are packaged, shipped, and stored in separate containers. According to one preferred method of treatment, the First Part and Second Part are mixed together at the site where treatment is needed and the mixture is applied to the substrate in need of treatment. According to another preferred method of treatment, the First Part and Second Part are applied to substrate in need of treatment substantially simultaneously (such as, by two separate liquid streams from a squeeze bottle at the same time or by using a bottle that combines two liquid streams into a single stream) or sequentially (such as by sprinkling the Dry Second Part on the stain and then pouring or spraying the First Part on the stain) so that both parts are active in treating the stain or odor at the same time without having been pre-mixed. By pre-mixing or applying both parts to treat a substrate at the same time, a two part treatment is created on site.

According to one preferred embodiment, Part One and Dry Part Two of the treatment formula are packaged, shipped, and sold in separate compartments, and combined together, preferably in a single bottle or container by the user at the site of the stain or odor being treated. The First Part with Dry Part Two mixture is created by adding the Dry Part Two powder directly into the Part One in a bottle or container (preferably the bottle or container in which the First Part was shipped and sold). Preferably, around 0.48-0.58 grams of the Dry Part Two is added in-situ to around 8-12 ounces of Part One to create the two part treatment composition. Most preferably around 0.52 grams of Dry Part Two is added to 12 ounces of Part One. Once Dry Part Two is combined with Part One, the two part treatment is applied to an organic matter stain or spill and the bacteria spores will germinate and grow utilizing organic food sources from the spill or stain.

According to another preferred embodiment Part One and Liquid Part Two are filled (packaged) into two separate bottles that are preferably connected or attached together or otherwise sold together, but a single bottle with a divider to separate the two parts may also be used. Any known bottle configuration may be used provided it keeps the Part One and Liquid Part Two compositions from contacting each other prior to the time they are to be used together to treat a stain or odor. Most preferably, the two bottles are configured to lock together, such as with a top or lid that securely fits onto both bottles to lock them together. The two treatment formulas in the locking bottles are then labeled, packaged, shipped and sold as one unit. When the two part bottle is squeezed, most preferably, the top, lid, or nozzle is configured to mix the Part One and Liquid Part Two into a single stream of treatment composition in-situ that is applied directly to the treatment area. A lid or nozzle configuration that has two separate streams that may be applied simultaneously or in substantially immediate sequential succession to the treatment area may also be used.

### BRIEF DESCRIPTION OF THE DRAWINGS

The embodiments described herein may be better understood by referring to the following description in conjunction with the accompanying drawings.

FIG. 1 is an exemplary embodiment of a carpet square post treatment with the 2-part mixture after 24 hour incubation of the carpet square on TSA plate in accordance with the present disclosure.

FIG. 2 is an exemplary embodiment of growth from carpet pile is readily seen and not obscured by the carpet square in accordance with the present disclosure.

FIG. 3 is an exemplary embodiment of growth from carpet square at Time 0 immediately after treatment in accordance with the present disclosure.

FIG. 4 is an exemplary embodiment of growth from carpet square at 2 Hours after treatment in accordance with the present disclosure.

FIG. 5 is an exemplary embodiment of growth from carpet square at 4 Hours after treatment in accordance with the present disclosure.

FIG. 6 is an exemplary embodiment of growth from carpet square at 6 Hours after treatment in accordance with the present disclosure.

FIG. 7 is an exemplary embodiment of growth from carpet square at 24 Hours after treatment in accordance with the present disclosure.

### DETAILED DESCRIPTION OF THE DISCLOSURE

A stain and odor treatment composition and system according to one preferred embodiment of the invention comprises a two part formula, a Part One liquid composition containing an oxidizer and a Liquid Part Two liquid composition comprising Bacillus spores or a Dry Part Two powder composition comprising Bacillus spores. When the two parts are combined together by the user in-situ, the two parts combine to form the final treatment composition. By combining the parts in-situ, the beneficial use of both an oxidizer and a Bacillus spores treatments, in combination, are obtained, which would not be possible if the parts were pre-mixed prior to commercial sale since the Bacillus spores would no longer be viable once it reached the consumer and point of application given the amount of time the Bacillus spores would be in contact with the oxidizer in a pre-mixed formula.

According to one preferred embodiment, Part One comprises water and an oxidizer and optionally comprises a fragrance and a surfactant. Preferably, Part One of the formula comprises 90-99% water and 0.15-2.5% hydrogen peroxide (27.5% active), and optionally 0.10-0.75% surfactant and 0.01-0.20% fragrance. Most preferably, Part One of the formula comprises 96-99% water, 0.20-2.5% hydrogen peroxide (27.5% active), 0.20-0.50% surfactant, and 0.03-0.07% fragrance. These percentages are by weight. If an optional surfactant is used, it is preferably a negatively charged surfactant, such as sodium lauryl sulfate, which allows osmotic inhabitation to occur for continuation of cleaning and detergent activity on soils and odors. Other surfactants may also be used. The hydrogen peroxide used is preferably cosmetic grade, but other solutions may be used with modifications to the amount of ingredients in the First Part of the formula as will be understood by those of ordinary skill in the art.

The Second Part of the formula may comprise a Liquid Part Two or a Dry Part Two, both of which comprise one or more species of Bacillus in spore form. Spore-forming Bacillus species that may be used include, but are not limited to, B. amyloliquefaciens, B. clausii, B. circulans, B. coagulans, B. firmus, B. lactis, B. laterosporus, B. laevolacticus, B. lentus, B. licheniformis, B. megaterium, B. mucilaginosus, B. mycoides, B. olymyxa, B. polyfermenticus, B. pumilus. B. simplex, B. sphaericus, B. subtilis. Those skilled in the art will know that any Bacillus species that is spore-former competent may also be used. More preferably, at least one of the species used in the Second Part is B. subtilis spores because they are generally more resistant to hydrogen peroxide exposure than other Bacillus species. Preferably, the Second Part comprises B. subtilis, B. licheniformis, and B. megaterium, and optionally B. pumilus. Most preferably, the Second Part comprises two B. subtilis strains, two B. licheniformis strains, one B. megaterium strain, and one B. pumilus strain.

According to one preferred embodiment, Liquid Part Two is a Bacillus spore treatment which comprises water, a preservative, one or more species of Bacillus spores, and a surfactant; and may optionally contain alcohol and fragrance. Preferably, Liquid Part Two comprises 90-99% water, 0.05-0.5% preservative, 0.1-0.7% Bacillus spores, and 0.1-0.5% surfactant. Optionally, the Liquid Second Part may contain 0.1-1.0% fragrance and 0.5-3% alcohol. Most preferably, the Liquid Second Part of the formula comprises around 98.1111% water, around 0.1% preservative, around 0.3867% Bacillus spores, around 0.2% surfactant, around 0.05% fragrance, and may contain around 1.0-2.5% alcohol. These percentages are by weight.

In another preferred embodiment, Dry Part Two comprises a dry Bacillus spore powder, which may be packaged together with sodium bicarbonate, which has several functions, i.e., a diluent, a pH buffer, or a deodorizer; and a powder lubricant. Preferably, Dry Part Two comprises 10-30% of one or more species of Bacillus spores, 70-90% sodium bicarbonate, and 0.1-1.0 powder lubricant. Most preferably, Dry Part Two comprises 14.5% bacterial spores, 84.5% sodium bicarbonate, and 0.5% powder lubricant. These percentages are by weight.

According to one preferred embodiment, a treatment system according to the invention comprises two containers, one for holding Part One and the other for holding Part Two of the composition separate from the Part One during shipment and storage. The two parts are then mixed together at the point of use to treat an area having a stain containing organic matter or an odor caused by organic matter. According to another preferred embodiment, a treatment system comprises a single container that is divided or compartmentalized to maintain Part One and Part Two separate from each other so they do not contact each other until the time of application to a treatment area.

According to one preferred embodiment of a treatment system, Part One and Liquid Part Two are filled (packaged) into two separate bottles that are sold together so that the two parts remain separate until the contents from both bottles are applied to the treatment area. According to another preferred embodiment, the two separate bottles may be connected together in various configurations allowing the contents of the bottles to be applied simultaneously or substantially simultaneously. In one embodiment, the two bottles are locked together by a single top. After filling, the top used for the locking bottles securely fits onto both bottles. The two treatment formulas in the locking bottles are then labeled, packaged, shipped and sold as one unit. When the two part bottle is squeezed, preferably a top, lid, or nozzle releases a stream of Part One and a stream of Liquid Part Two, and the two streams are mixed together to form one treatment composition in-situ. The mixed stream is applied directly to the treatment area. Separate streams of the Part One and Liquid Part Two that are applied substantially simultaneously or in substantially immediate sequential succession to a treatment area may also be used.

According to another preferred embodiment of a treatment system, when using the Part One and Dry Part Two treatment mixture, the Part One is packaged and sold in a bottle or container that holds around 8-12 ounces. A smaller sized bottle or container is preferred since the formula is designed for single-use applications, so that the entire contents of the bottle, once Part One and Dry Part Two are combined, would be applied to the treatment area. This limits the exposure time of the Bacillus spores to the hydrogen peroxide in the product. As the Bacillus spores in the formula will not survive long term exposure to the oxidizing agent, any treatment mixture not used soon after the two parts are combined would not be as effective. The size of the bottle or container in which Part One is packaged preferably provides sufficient space in the bottle or container to allow Dry Part Two to be added to Part One in-situ by the end-user. In one preferred embodiment, Dry Part Two may be separately contained in a pouch or other suitable container packaged and sold along with the bottle containing Part One of the treatment mixture, so that it is easily opened and emptied into the bottle containing the Part One of the treatment mixture. In another preferred embodiment, the Dry Part Two is sealed in a compartment or storage area within the lid or cap of the bottle containing the First Part of the formula. When ready for use, the user would remove or puncture the seal on the lid or cap (or open the separate pouch or container); and empty the Dry Part Two mixture into the bottle containing the First Part to combine the two parts of the formula. Most preferably, the bottle or container is shaken vigorously to mix the two parts of the formula prior to applying it by spraying or pouring onto the treatment area containing the spill or stain or malodorous spot.

The hydroxyl radicals react very quickly when the spores are added to the product. The contact time is not long enough for the hydroxyl radicals to inactivate or kill the spores. The short-lived, free, hydroxyl radicals have a half-life of approximately 10⁻⁹ seconds.

Testing has shown that the Dry Part Two Bacillus spores added to the Part One are not inactivated or killed by exposure to hydrogen peroxide and that the Bacillus spores are viable to germinate, and capable of growth and degrading organic matter for at least 24 hours after mixing the 2-part treatment and applying to carpet.

A treatment formula according to a preferred embodiment of the invention where Part One was mixed with Dry Part Two was tested on a 10×10 inch carpet sample to determine the viability of the spores after coming into contact with the oxidizing agent. A growth medium was applied to the carpet sample to simulate organic soils, spills, or stains. Once the treatment formula is applied to the carpet samples, the hydrogen peroxide foamed upon reaction with the organic growth medium, but the foaming dissipated after a few minutes. Rapid and vigorous reaction of the hydrogen peroxide with the organic matter is considered to negatively impact spore viability. However, these tests showed that the bacteria spores survived in the bottle after coming into contact with the hydrogen peroxide and on the carpet samples after the reaction between the hydrogen peroxide and the organic matter in the simulated spill.

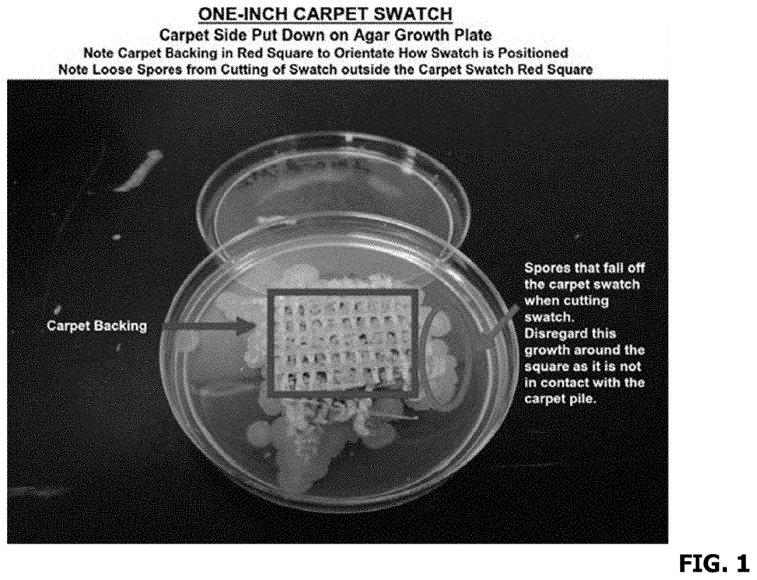

Small sections of the carpet, approximately 1×1 inch, were periodically cut away after application of the treatment composition. Time “0” samples were immediately cut. The remainder of the carpet sample was covered with a sheet of foil, and placed in an incubator. Additional sections of the carpet were cut at 2 hours, 4 hours, 6 hours, and 24 hours after application of the treatment composition. FIG. 1 illustrates how a carpet square was initially placed on the face of a TSA plate with the carpet pile side down onto the plate surface. Note that carpet backing obscures the growth that may be under the carpet square. The plates were incubated for a total of 48 hours. The amount of growth or no growth on the plate would indicate if the spores were killed or inactivated at any time during 24 hours incubation on the carpet sample.

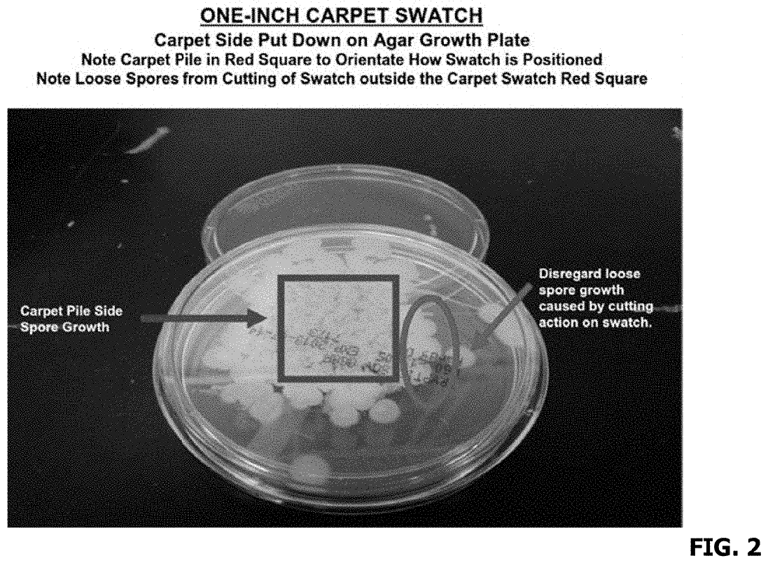

FIG. 2 illustrates a change in procedure where the carpet square, pile side down, was removed from the plate surface after one hour. The plate incubation was continued for a total of 48 hours. The amount of growth or no growth on the plate indicates if the spores were killed or inactivated at any time during 24 hours incubation on the carpet sample. Any growth that was obscured by the carpet square is now visible to aid determination if the spores were still viable and able to grow.

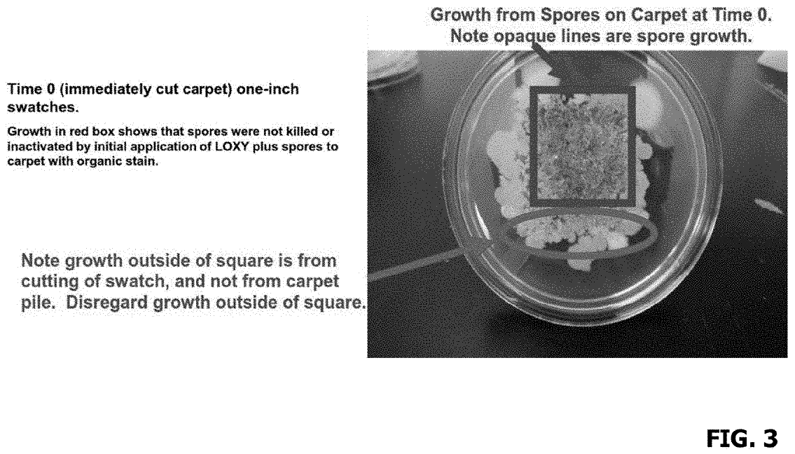

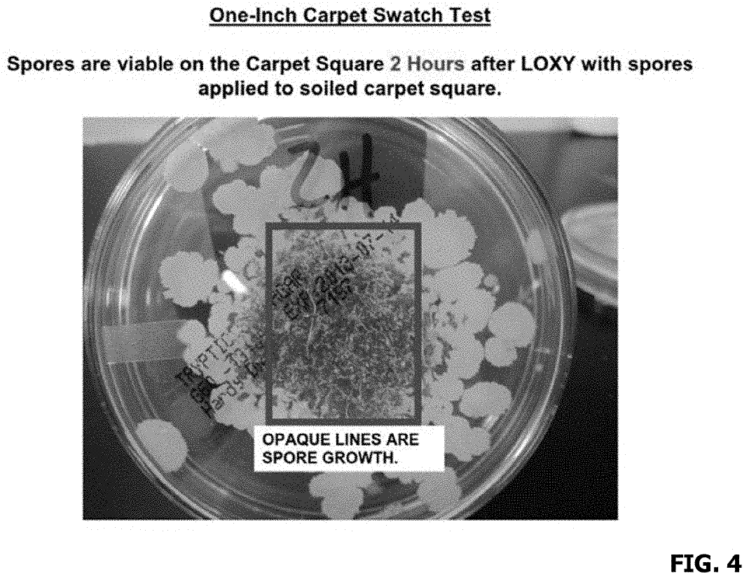

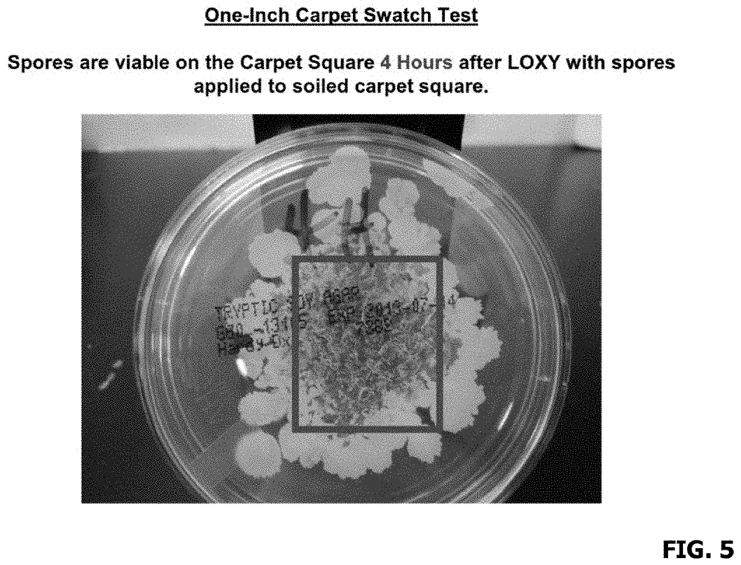

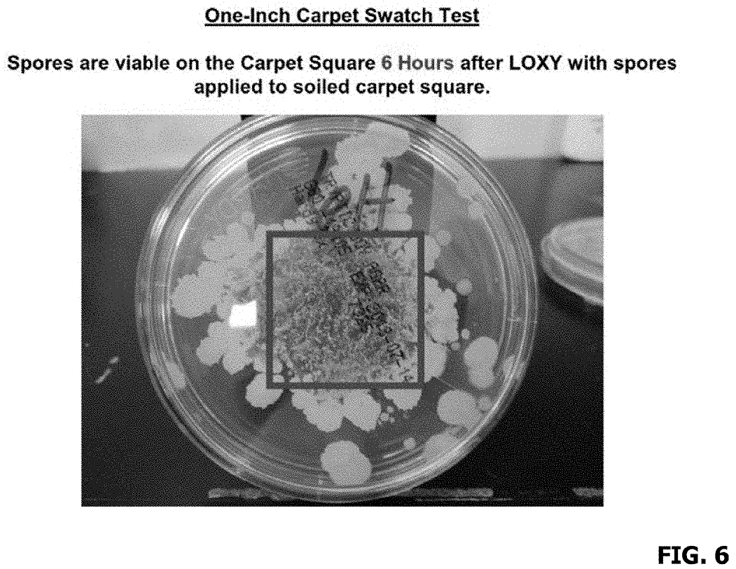

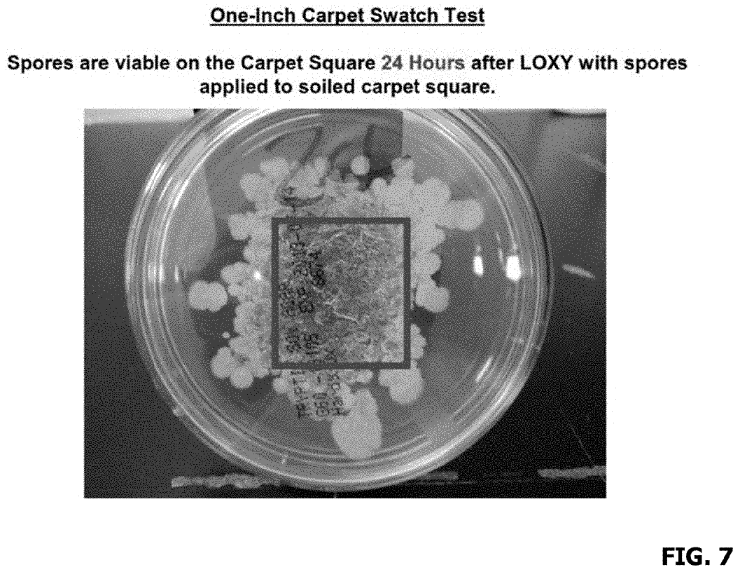

FIG. 3 shows the viability of the spores via growth from the Time 0 square. Growth inside the box shows that the spores were not killed by the mixing of the 2-part treatment, and the spores were not killed during and after the application of the 2-part treatment composition to the carpet. The opaque lines show spore growth. The growth outside the square (shown in the oval) is from cutting of the swatch and should be disregarded. FIG. 4 through FIG. 7 show that the spores applied to the carpet via the 2-part treatment did not die while on carpet after 2 hours, 4 hours, 6 hours, or 24 hours. Demonstrating that the Bacillus cells are viable, and capable of growth and degrading organic matter for at least 24 hours after mixing the 2-part treatment.

Spore counts were performed on the 2-part mixture immediately after mixing the parts together which is the Time 0 count. The 2-part mixture was retained, and set at Room Temperature (75° C.). Additional counts were done on the mixture at 2, 4, 6, and 24 hours post mixing the parts together. There was a decrease in count over the 24 hours, but the 24 hour decrease was only approximately 35% decrease (not kill) as shown in Table 1.

TABLE 1
Standard, heterotrophic plate counts on TSA were performed on the
2-part and stability of the bacteria spores when mixed with the
hydrogen peroxide containing liquid from Time 0 until 24 hours.

| | BACILLUS SPORES PER ML OF |
|---|---|
| SAMPLE TIME AFTER | LOXY CONTAINING DRY SPORE |
| ADDITION OF SPORE POWDER | POWDER (Bacillus spores per ml) |
| Time 0 | 3.7 × 10⁸ |
| Time 0 - Duplicate Count | 3.9 × 10⁸ |
| 2 Hour | 3.2 × 10⁸ |
| 2 Hour - Duplicate Count | 3.5 × 10⁸ |
| 4 Hour | 3.8 × 10⁸ |
| 4 Hour - Duplicate Count | 3.3 × 10⁸ |
| 6 Hour | 3.7 × 10⁸ |
| 6 Hour - Duplicate Count | 3.4 × 10⁸ |
| 24 Hour * | 1.3 × 10⁸ |
| 24 Hour Count - Duplicate Count * | 1.3 × 10⁸ |

Based on this testing, the spores in the two part treatment composition will remain active and viable in the bottle (after mixing with the First Part of the formula containing the hydrogen peroxide) for at least 24 hours and will remain active and viable for at least 24 hours after the treatment composition is applied to an organic spill or stain. However, it is preferred to apply the treatment composition to the treatment area soon after mixing of the two parts, preferably within 30 minutes of mixing, to get the full effect of the combined treatment.

Other embodiments of the treatment formula were also tested using varying amounts of hydrogen peroxide. The spore survival evaluations at 5 minutes, 2 hours, 4 hours, 6 hours, and 24 hours after mixing the spores with the hydrogen peroxide are shown in Table 2 below.

TABLE 2
Time of Bacillus spore survival after 2-part mixture with Part
One containing different amounts of hydrogen peroxide (H₂O₂).

| | Spore | Spore | Spore | Spore | Spore |
|---|---|---|---|---|---|
| Percent | Survival | Survival | Survival | Survival | Survival |
| H₂O₂ | 5 Minutes | 2 Hours | 4 Hours | 6 Hours | 24 Hours |
| 1.43 | 0 | 0 | 0 | 0 | 0 |
| 0.71 | Excellent | Good | Fair | Fair | Poor |
| 0.285 | Excellent | Excellent | Excellent | Excellent | Excellent |

These results indicate that the spores are viable for significant periods of time after mixing with higher concentrations of hydrogen peroxide, but that at a 1.43% hydrogen peroxide level the spores were not viable.

A preferred method of treating a stain or odor comprises providing an oxidizer part and a bacteria spores part; mixing the parts together at the location of the area having a stain or odor to be treated to form an in-situ treatment composition; and applying the treatment composition to the area with the stain or odor. Most preferably, the treatment composition is one according to invention described herein and other ingredients, such as a surfactant or fragrance, may also be added either at the treatment location or with one or more of the other ingredients at the place of production prior to commercial sale. After mixing, the bacteria spores remain viable for at least two hours, and may be viable for up to or longer than 24 hours. The user preferably periodically checks the treatment area after application of the treatment composition and may blot the area to test the effectiveness of removing the stain or odor.

Once satisfied with the level of treatment, the user may further blot the area to remove excess treatment composition and aid in drying the treatment area. After a period of time, usually at least 24 hours depending on the formulation of the treatment composition, once the bacteria are no longer viable to effectively treat the stain or odor, the user may blot or dry the treatment area and repeat the steps with a newly mixed batch of treatment composition. It is the preferred process that the composition be applied to the treatment area within 30 minutes, and more preferably within 10 minutes, of mixing Part One with Part Two of the composition for Part One to be at its peak performance.

Those of ordinary skill in the art will also appreciate upon reading this specification and the description of preferred embodiments herein that modifications and alterations to the device may be made within the scope of the invention and it is intended that the scope of the invention disclosed herein be limited only by the broadest interpretation of the appended claims to which the inventors are legally entitled.

What is claimed is:

1. A composition for treating an area having a stain containing organic matter or an odor caused by organic matter, the composition comprising:
a first part comprising an oxidizing agent; and
a second part comprising Bacillus spores,
wherein at least some of the Bacillus spores remain viable and germinate into Bacillus vegetative cells that continue to be capable of growth on the organic matter for at least six hours after the first and second parts are combined together and applied to the area.

2. The composition according to claim 1 wherein the first part comprises about 0.15-2.5% hydrogen peroxide (27.5% active) and about 90-99% water, by weight; optionally a surfactant; and optionally a fragrance.

3. The composition according to claim 2 wherein the first part further comprises about 0.20-0.50% surfactant.

4. The composition according to claim 3 wherein the surfactant is an anionic surfactant, sodium lauryl sulfate or SDS or other SLS derivatives.

5. The composition according to claim 3 wherein the first part further comprises about 0.03-0.07% fragrance.

6. The composition according to claim 1 wherein at least some of the Bacillus spores remain viable and capable of growth for at least 24 hours after the first and second parts are combined together and applied to the area.

7. A composition for treating an area having a stain containing organic matter or an odor caused by organic matter, the composition comprising: an oxidizing agent and Bacillus spores.

8. The composition according to claim 7 wherein the Bacillus spores are viable to germinate for at least 6 hours after applying to the area and capable of growth and degrading organic matter for at least 24 hours applying to the area.

9. The composition according to claim 7 wherein the oxidizer is hydrogen peroxide and the Bacillus spores are one or more of B. amyloliquefaciens, B. clausii, B. circulans, B. coagulans, B. firmus, B. lactis, B. laterosporus, B. laevolacticus, B. lentus, B. licheniformis, B. megaterium, B. mucilaginosus, B. mycoides, B. olymyxa, B. polyfermenticus, B. pumilus, B. simplex, B. sphaericus, and B. subtilis.

10. The composition according to claim 9 wherein the hydrogen peroxide in an aqueous solution comprising about 0.15-2.5% (27.5% active) hydrogen peroxide.

11. The composition according to claim 10 wherein the Bacillus spores are in a dry powder form or an aqueous solution.

12. The composition according to claim 11 wherein the dry powder form comprises around 10-30% Bacillus spores, around 70-90% sodium bicarbonate, and around 0.1-1% powder lubricant.

13. The composition according to claim 11 wherein the Bacillus aqueous solution comprises around 90-99% water, around 0.05-0.5% preservative, around 0.1-0.7% Bacillus spores, around 0.1-0.5% surfactant, and optionally around 0.1-1% fragrance and optionally around 0.5-3% alcohol.

14. A method of treating an area having a stain containing organic matter or an odor caused by organic matter, the method comprising:
providing an oxidizer solution in a first container;
providing one or more species of Bacillus spores in a second container;
mixing the oxidizer solution and Bacillus spores at a location having the area in need of treatment to form an in-situ treatment composition; and
applying the composition to the area to be treated,
wherein at least some of the Bacillus spores are not killed or inactivated after mixing with the oxidizer solution.

15. The method of claim 14 wherein the oxidizer solution comprises water and hydrogen peroxide and the Bacillus spores comprise one or more of B. amyloliquefaciens, B. clausii, B. circulans, B. coagulans, B. firmus, B. lactis, B. laterosporus, B. laevolacticus, B. lentus, B. licheniformis, B. megaterium, B. mucilaginosus, B. mycoides, B. olymyxa, B. polyfermenticus, B. pumilus, B. simplex, B. sphaericus, and B. subtilis.

16. A method of treating a local area having a stain or odor, the method comprising:
providing a sealable first container and a sealable second container, each comprising a body and a lid, wherein at least a portion of one of the containers is configured to lock fit to at least a portion of the other container to lock the containers together, wherein the first container contains a liquid oxidizer composition and the second container contains a liquid Bacillus spore composition that does not contact the liquid oxidizer composition inside the locked together containers; and
squeezing the locked together containers simultaneously to release a stream of the oxidizer composition and a stream of the liquid Bacillus spore composition with the two streams mixing together outside of the containers; odor; applying the mixed stream to the local area having the stain or wherein the Bacillus spores remain viable and capable of germination for at least 6 hours after the mixed stream is applied to the local area and the germinated Bacillus spores remain viable and capable of growth for at least 24 hours after the mixed stream is applied to the local area.

17. A method of treating a local area having a stain or odor, the method comprising:
providing a first container comprising a body and a lid, the first container containing a liquid oxidizer composition;
providing a second container containing a dry powder Bacillus spore composition;
opening the first and second containers;
adding the dry powder Bacillus spore composition from the second container to the first container to form a liquid treatment composition;
applying the liquid treatment composition to the local area in need of treatment; and
wherein the Bacillus spores remain viable and capable of germination for at least 6 hours after the liquid treatment composition is applied to the local area and the germinated Bacillus spores remain viable and capable of growth for at least 24 hours after the liquid treatment composition is applied to the local area.

18. The method of claim 17 wherein the second container comprises the lid of the first container and a seal that keeps the dry powder Bacillus spore composition from contacting the liquid oxidizer composition prior to the opening and adding steps and wherein adding the dry powder comprises removing or puncturing the seal on the lid.

19. The method of claim 17 wherein the second container contains around 0.48-0.58 grams of the dry powder Bacillus spore composition and the first container contains around 8-12 ounces of the liquid oxidizer composition.

20. A system for treating an area having a stain containing organic matter or an odor caused by organic matter, the system comprising:
a first container containing a liquid oxidizer composition;
a second container containing a Bacillus spore composition; and
wherein at least some of the Bacillus spores remain viable and germinate into Bacillus vegetative cells that continue to be capable of growth on the organic matter for at least six hours after a liquid treatment composition of the liquid oxidizer composition and the Bacillus spore composition is applied to the area having the stain or odor.

21. The system of claim 20 wherein at least a portion of one of the containers is configured to lock fit to at least a portion of the other container to lock the first and second containers together;
wherein the Bacillus spore composition is a liquid that does not contact the liquid oxidizer composition inside the locked together containers; and
wherein the containers are configured to mix a stream of the liquid oxidizer composition discharged from the first container and a stream of the liquid Bacillus spore composition discharged from the second container when squeezed.

22. The system of claim 20 wherein the Bacillus spore composition is a dry powder, wherein the first container comprises a bottle and a lid, wherein the second container comprises the lid of the first container and a seal, and wherein the bottle is configured to receive a dry powder Bacillus spore composition from the second container to form a liquid treatment composition and the seal is configured to keep the dry powder Bacillus spore composition from contacting the liquid oxidizer composition prior to the dry powder being received in the bottle.
